(19) United States
(12) Patent Application Publication
(10) Pub. No.: US 2014/0186284 A1
(43) Pub. Date: Jul. 3, 2014

(54) CONDITIONING COMPOSITIONS FOR KERATINOUS SUBSTRATES

(71) Applicant: L'OREAL, Paris (FR)

(72) Inventors: Allen SHA, Edison, NJ (US); Dina J. BURAKOV, Millburn, NJ (US); Carmen CASTILLO BUCCI, Englewood, NJ (US)

(21) Appl. No.: 13/728,301

(22) Filed: Dec. 27, 2012

(51) Int. Cl. A61K 8/73; A61Q 5/06; A61Q 5/12

(52) U.S. Cl. CPC A61K 8/731; A61Q 5/12; A61Q 5/06
USPC 424/70.13

(57) ABSTRACT

The present invention is directed towards a conditioning composition comprising, in an aqueous medium, at least one nonionic film forming polymer; at least one viscosity modifying agent; at least one cationic polymer; at least one cationic surfactant; and wherein the composition is free of silicones. Preferably, the composition is clear in appearance. The present invention also relates to a process for conditioning keratinous substrates.

### FIELD OF THE INVENTION

The present invention relates to personal care and cosmetic compositions. In particular, the present invention relates to a composition and process for conditioning keratinous substrates wherein the composition contains a film forming polymer, a viscosity modifying agent, a cationic polymer and a cationic surfactant and is free of silicones.

### BACKGROUND OF THE INVENTION

Cosmetic and personal care products are available in various forms and one of the forms that are desired by many consumers is a clear aqueous product. At the same time, the consumer expects that such a product will provide desirable cosmetic benefits to keratinous substrates such as hair and skin.

Further, consumers prefer products that can serve more than one function or provide more than one cosmetic benefit. For example, with respect to hair care, consumers generally prefer a product that can clean and condition their hair in a single step. Consumers also prefer hair care products that can clean and allow the hair to be styled without the need for additional styling products. At the same time, consumers desire to use products that have unique, aesthetically pleasing appearances and/or textures, such as clear/transparent appearance.

Achieving multiple functionalities, however, can present formulation challenges because they generally require the introduction of ingredients that could affect the stability, appearance, and texture of the products as well as delivery of benefits to keratinous substrates such as hair and skin. For instance, a sufficient amount of such oils is required to condition hair or skin. However, it has been difficult to provide personal care compositions that incorporate such amounts so that the compositions remain stable, while remaining clear.

Certain water-insoluble ingredients, which are oftentimes desirable for the treatment and conditioning of keratinous substrates, are inherently difficult to incorporate into aqueous systems, such as shampoos, conditioners and skin care compositions, without forming a traditional emulsion in either cream or lotion form. Moreover, oftentimes, the presence of conditioning ingredients at levels that would impart appreciable cosmetic benefits to hair or skin and/or properties to cosmetic and personal care products result in unstable formulations resulting in undesirable phase separations in aqueous systems. Furthermore, such ingredients could affect the viscosity or rheology and appearance of the cosmetic compositions such that they are less desirable to the consumers.

One way of alleviating the formulation problems encountered with including conditioning agents into cosmetic compositions is the use of polydimethylsiloxanes, commonly known as silicones. For example, these silicone-based conditioning agents, such as dimethicone and cyclodimethicone, have been popular in hair care products because they are useful in making hair look full, shiny, smooth and easy to comb. However, upon prolonged usage, silicone-based conditioning agents may form a coating on the hair, thus preventing the hair from getting sufficient moisture or from being adequately cleansed. Therefore, despite their apparent effects in making hair look full and shiny, conditioning formulations that contain silicon-based conditioning agents raise concerns among consumers.

Thus, various existing technologies have been developed to address the problems described above and achieve certain cosmetic attributes.

For example, US2004234491 discloses a transparent gel type hair conditioner of low viscosity that employs Polyquaternium-37 and solubilizers such as nonionic, cationic and amphoteric surfactants, and is free of fatty alcohols. U.S. Pat. No. 4,610,874 discloses a clear base composition for a hair conditioner which uses a derivative of ethoxylated/acetylated lanolin, an ionic polymer, and hydroxyethylcellulose. WO0040207 concerns a transparent conditioner composition containing a breakable visible particle which is comprised of polysaccharides and/or saccharides and their derivatives and viscofying agents. US2005002892 discloses a conditioning composition that employs a cationic crosslinked thickening polymer and a surfactant in order to achieve a suitable rheology; US2003216267 discloses a transparent concentrated hair conditioning composition of a particular turbidity and comprising a conditioning agent chosen from cationic surfactants, cationic polymers, silicone compounds and polyakylene glycols. US2004247550 and US2005175568 disclose hair or skin conditioning compositions that employ surfactants, a hydrophobically modified cationic thickening polymer and a silicone such as a hydrophobically modified silicone.

Nevertheless, many challenges still exist, not only in effectively introducing ingredients into products used to condition hair and skin, but also in creating products which provide several benefits to hair and skin while having the appearance and texture that are desirable to the consumer.

Thus, the ability to provide compositions for application onto keratinous substrates which can provide multiple benefits, including good conditioning, light-weight feel, and desirable cosmetic attributes to the keratinous substrates, remain as additional areas for improvement. It is also desirable to provide methods of conditioning keratinous substrates with such compositions and methods of making these compositions.

### SUMMARY OF THE INVENTION

The present disclosure is directed to a conditioning composition containing, in an aqueous medium:
(a) at least one nonionic film forming polymer;
(b) at least one viscosity modifying agent;
(c) at least one cationic polymer;
(d) at least one cationic surfactant; and
wherein the composition is free of silicones.

The above-described composition is preferably clear in appearance.

The above-described composition is also preferably free of fatty alcohols.

The present invention further relates to a process for conditioning a keratinous substrate involving contacting keratinous substrates with the above-described composition.

It has been surprisingly and unexpectedly discovered that the application of the above-described composition onto keratinous substrates, such as hair and skin, provides excellent conditioning, delivery of active ingredients, smoothness, and softness in the absence of silicones. In particular, light-weight conditioning effects were achieved. When the keratinous substrate is hair, the above-described composition surprisingly and unexpectedly also provides good styling effects, good manageability, and a good/sleek feel to the hair.

It was also surprisingly and unexpectedly found that the compositions of the present disclosure are stable and have a desirable viscosity or rheology even in the absence of silicones and fatty alcohols that allows for controlled dispensing or pouring of the desired amount onto keratinous substrates, while still being easy to spread on, work through, and rinse from the keratinous substrates.

### DETAILED DESCRIPTION

The term “comprising” (and its grammatical variations) as used herein is used in the inclusive sense of “having” or “including” and not in the exclusive sense of “consisting only of”. The terms “a” and “the” as used herein are understood to encompass the plural as well as the singular.

Other than in the operating examples, or where otherwise indicated, all numbers expressing quantities of ingredients and/or reaction conditions are to be understood as being modified in all instances by the term “about,” meaning within 10% to 15% of the indicated number.

“Film former” or “film forming agent” or “film forming polymer” as used herein means a polymer or resin or material that leaves a film on the substrate to which it is applied, for example, after a solvent accompanying the film former has evaporated, absorbed into and/or dissipated on the substrate.

“At least one” as used herein means one or more and thus includes individual components as well as mixtures/combinations.

The term “clear” as used herein also means “transparent” or “translucent” such that a person is able to see through the composition with their naked eye. The term “clear” as used herein also means that the composition does not exhibit phase separation.

The term “clear” or “transparent” or “translucent” as used herein can also mean that the composition has visible light transmittance values of about 60% or more, or more preferably, about 70% or even more preferably, about 80% or more. The transmittances can be measured at a particular wavelength, such as at about 700 nm, and through a 1 cm thick sample, using a commercial UV-visible spectrophotometer. The term “clear” or “transparent” or “translucent” as used herein is not meant to encompass those compositions which a person cannot see through with their naked eye such as those which are pearlescent, frosted, vey hazy, opaque, or very cloudy in appearance.

In preferred embodiments, the composition of the present disclosure has the % transmittance described above for at least one month, more preferably, for at least three months, and even more preferably, for at least a year at 25° C., following preparation of the composition.

The term “free of silicones” as it is used herein means that while it is preferred that no silicones be present in the composition, it is possible to have very small amounts of silicones in the compositions of the invention provided that these amounts do not materially affect at least one, preferably most, of the advantageous properties of the conditioning compositions of the invention. In particular, “free of silicones” means that silicones can be present in the composition at an amount of less than about 0.25% by weight, typically less than about 0.1% by weight, typically less than about 0.05% by weight, and more typically 0% by weight, based on the total weight of the composition as a whole.

The silicones in the term “free of silicones” as used herein refers to those silicone compounds that are silicone-based conditioning agents.

The term “free of fatty alcohols” as it is used herein means that while it is preferred that no silicones be present in the composition, it is possible to have very small amounts of fatty alcohols in the compositions of the invention provided that these amounts do not materially affect at least one, preferably most, of the advantageous properties of the conditioning composition of the invention. In particular, “free of fatty alcohols” means that fatty alcohols can be present in the composition at an amount of less than about 0.2% by weight, typically less than about 0.1% by weight, typically less than about 0.05% by weight, and more typically 0% by weight, based on the total weight of the composition as a whole.

The term “fatty alcohols” as used herein refers to those compounds that are employed in conventional conditioning compositions, such as hair conditioners, which are typically in emulsion form.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

### Nonionic Film Forming Polymer

The non-ionic fixing polymers which can be used according to the present invention are chosen, for example, from: vinylpyrrolidone homopolymers; copolymers of vinylpyrrolidone and of vinyl acetate; polyalkyloxazolines, such as the polyethyloxazolines provided by the company Polymer Chemistry Innovations under the names Aquazol® HP, and Aquzol® HVIS; vinyl acetate homopolymers, such as the product provided under the name UCAR™ 130 Latex Resin by the company Dow Chemical or the product provided under the name Ultrapure Polymer 2041-R 012 by the company Ultra Chemical, Inc.; copolymers of vinyl acetate and of acrylic ester, such as the product provided under the name Rhodopas AD 310 from Rhone-Poulenc; copolymers of vinyl acetate and of ethylene, such as the product provided under the name Dermacryl® LOR by the company Akzo Nobel; copolymers of vinyl acetate and of maleic ester, for example of dibutyl maleate, such as the product provided under the name Appretan MB Extra by the company Clariant; copolymers of polyethylene and of maleic anhydride; alkyl acrylate homopolymers and alkyl methacrylate homopolymers, such as the product provided under the name Micropearl RQ 750 by the company Matsumoto or the product provided under the name Luhydran® A 848 S by the company BASF; acrylic ester copolymers, such as, for example, copolymers of alkyl acrylates and of alkyl methacrylates, such as the product provided by the company Dow Chemical under the name Primal™ AC-261 K and the product provided by Evonik under the name Eudragit® NE 30 D, by the company BASF under the names Acronal® 601, Luhydran® R 8833 or 8845, or by the company Clariant under the names Appretan® N 9213 or N9212; copolymers of acrylonitrile and of a non-ionic monomer chosen, for example, from butadiene and alkyl(meth)acrylates; mention may be made of the products provided under the names Nipol LX 531 B by the company Nippon Zeon or those provided under the name CJ 0601 B by the company Rohm and Haas; polyurethanes, such as the products provided under the names Acrysol™ RM 1020 or Acrysol™ RM 2020 by the company Dow Chemical or the products Uraflex XP 401 UZ or Uraflex XP 402 UZ by the company DSM Resins; copolymers of alkyl acrylate and of urethane, such as the product 8538-33 by the company National Starch; and polyamides, such as the product Estapor LO 11 provided by the company Rhone-Poulenc.

Other suitable examples of film forming polymers are fixing polymers of grafted silicone type comprising a polysiloxane portion and a portion composed of a non-silicone organic chain, one of the two portions constituting the main chain of the polymer and the other being grafted onto the said main chain. These polymers can be non-ionic.

According to preferred embodiments, the at least one nonionic film forming polymer is not contained in an emulsion composition, i.e., it is not pre-emulsified, before incorporating said film forming polymer into the compositions of the present disclosure.

Preferred nonionic film forming polymers of the present disclosure are chosen from vinylpyrrolidone homopolymers and copolymers of vinylpyrrolidone and of vinyl acetate. Vinylpyrrolidone homopolymers (INCI name: polyvinylpyrrolidone) are commercially available from Ashland Specialty Ingredients under the tradename PVP K. Copolymers of vinylpyrrolidone and of vinyl acetate (INCI name: VP/VA copolymer) are commercially available from BASF under the tradename Luviskol® VA.

The at least one nonionic film forming polymer is present in the composition in an amount of from about 0.05 to about 2% by weight, such as from about 0.1 to about 1.5% by weight, and from about 0.5 to about 1% by weight, including all ranges and subranges there-between, based on the total weight of the composition.

### Viscosity Modifying Agent

The viscosity modifying agents of the present disclosure include, but are not limited to, anionic or nonionic polysaccharide polymers such as gum tragacanth, sodium or propylene glycol alginate, kappa-, iota-,or lambda-carrageenan, guar or hydroxyl propyl guar gum, karaya gum, gum arabic, locust bean gum, konjac mannan gum, gellan, xanthan, succinoglycan or its acidic or enzymatic hydrolysates, sodium carboxymethyl cellulose, methycellulose, hydroxylethylcellulose, hydroxypropylmethylcellulose, and hydroxypropylcellulose; and/or hydrophobically modified anionic, cationic, or nonionic polymers such as, but not limited to, alkyl and/or substituted hydroxyethylcellulose, lauryl dimethyl ammonium substituted epoxide of hydroxyethylcellulose, propoxylated cellulosic, xanthan, succinoglycan, or polygalactomannoses, alkyl methacrylates/crosslinked acrylic acid copolymer and/or acrylonitrile/acrylates block copolymer.

In preferred embodiments, chemically modified or unmodified non-ionic guar gums may be used in the compositions of the present disclosure.

The unmodified non-ionic guar gums are, for example, the products sold under the name Vidogum GH by the company Unipectine and under the name Jaguar® S by the company Rhodia. The modified non-ionic guar gums, which can be used according to the invention, are preferably modified by C1-C6 hydroxyalkyl groups. Mention may be made, by way of example, of the hydroxymethyl, hydroxyethyl, hydroxypropyl, and hydroxybutyl groups. These guar gums are well known in the state of the art and can, for example, be prepared by reacting the corresponding alkene oxides, such as, for example, propylene oxides, with guar gum, so as to obtain a guar gum modified by hydroxypropyl groups.

Other nonionic film forming polymers may be chosen from non-ionic guar gums optionally modified by hydroxyalkyl groups are, for example, sold under the trade names Jaguar® HP8, Jaguar® HP60, Jaguar® HP120, and Jaguar® HP 105 by the company Rhodia or under the name Galactasol™ 4H4FD2 by the company Ashland Specialty Ingredients.

The alkyl radicals of the non-ionic fixing polymers have from 1 to 6 carbon atoms, unless otherwise mentioned.

Other examples of viscosity modifying agents include water swellable/soluble cationic polymers from quaternized polysaccharides such as trimethyl ammonium substituted epoxide of hydroxyethyl cellulose, diallyldimethyl ammonium salts of hydroxyethylcellulose, deacylated chitin or chitosan, dihydroxypropyl chitosan trimonium chloride, hydroxypropltrimethyl ammonium chloride guar, locust bean, or konjac mannan gum; quaternized synthetics such as acrylamide dimethyl diallyl ammonium chloride copolymers, acrylamide/dimethyl diallyl ammonium chloride/acrylic acid terpolymer, quaternized poly(vinyl pyrrolidone/dimethyl amino ethylmethacrylate), poly(vinylpyrrolidone/methacrylamidopropyl trimethylammonium chloride), polyvinyl pyrrolidone/methylvinylimidazolinium chloride or methyl sulfate copolymer, chloroethylether/dimethylaminopropylamine/adipate or azelate terpolymer, vinylpyrrolidone/methacrylamidopropyl trimethylammonium chloride, acrylonitrile/acrylic acid/dimethylpropanediammonium acrylates sulfate terpolymer.

The viscosity modifying agents of the present disclosure include, but are not limited to, polymers based on acrylic acid crosslinked with an allyl ether of pentaerythritol, or an allyl ether of sucrose, or an allyl ether of propylene. Such polymers are also known as gelling agents and are generally of high molecular weight. Variations of such copolymers are also termed carbomers. The carbomers may have high molecular weights ranging from about 700,000 to about 5,000,000.

Other viscosity modifying agents of the present disclosure also include carbomers which are commercially available under the tradename Carbopol® from the supplier, Lubrizol, examples of which are Carbopol® 934, 940, 941, 951, 954, 956, 980, 981, 1342, 2984, Carbomer EDT 2001, Carbomer-934P, and Carbomer Ultrez 10. Carbomers may also be commercially available under the tradename TEGO® Carbomer from the supplier, Evonik Industries, examples of which are TEGO® Carbomer 134, 140, 141, and 340 FD.

The at least one viscosity modifying agent of the present disclosure may further comprise other compounds to improve the viscosity modifying, gellifying and thickening properties of the viscosity modifying agent.

Preferred viscosity modifying agents of the present disclosure are chosen from hydroxyethylcellulose, commercially available from Ashland Specialty Ingredients under the tradenames Natrosol® 250 HHR PC, Natrosol® 250 HHR CS, Natrosol® 250 MR, Natrosol® 250 MR CS, and from Amerchol under the tradename Cellosize™ QP 4400 H.

Other preferred viscosity modifying agents of the present disclosure are chosen from cetyl hydroxyethylcellulose, commercially available from Ashland Specialty Ingredients under the tradename Natrosol® Plus 330, guar gum, commercially available from Ashland Specialty Ingredients under the tradename Supercol® U Guar Gum, and xanthan gum commercially available from Rhodia under the tradename Rhodicare® XC.

The at least one viscosity modifying agents is present in the composition in an amount of from about 0.05 to about 2% by weight, such as from about 0.1 to about 1.5% by weight, or from about 0.5 to about 1.2% by weight, or from about 0.5 to about 0.75% by weight, including all ranges and subranges there-between, based on the total weight of the composition.

### Cationic Polymers

The at least one cationic polymer of the present disclosure include, but are not limited to, hexadimethrine chloride, polyquaternium-4, polyquaternium-6, polyquaternium-7, polyquaternium-10, polyquaternium-11, polyquaternium-16, polyquaternium-22, polyquaternium-32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, and guar hydroxyproyltrimonium chloride.

Preferred cationic polymers of the present disclosure are chosen from polyquaternium-10, commercially available from Dow Chemical under the tradename Ucare™ Polymer JR 400, polyquaternium-70, commercially available from Croda under the tradename LUSTREPLEX™, and guar hydroxyproyltrimonium chloride, commercially available from Ashland Specialty Ingredients under the tradenames N-Hance™ CG and N-Hance™ 3196, and from Rhodia under the tradenames Jaguar® C-13-S and Jaguar® C17.

The at least one cationic polymer is present in the composition in an amount of from about 0.05 to about 5% by weight, such as from about 0.1 to about 4% by weight, or from about 0.3 to about 3% by weight, or from about 0.5 to about 2% by weight, or from about 0.5 to about 1% by weight, including all ranges and subranges there-between, based on the total weight of the composition.

In certain embodiments, the compositions of the present disclosure contain at least two cationic polymers.

In some embodiments, the at least two cationic polymers in the compositions of the present disclosure include polyquaternium-10 and polyquaternium-70.

In other embodiments, the at least two cationic polymers in the compositions of the present disclosure include polyquaternium-10 and guar hydroxyproyltrimonium chloride.

In yet other embodiments, the at least two cationic polymers in the compositions of the present disclosure are chosen from polyquaternium-10 and guar hydroxyproyltrimonium chloride, wherein polyquaternium-10 is present in an amount of from about 0.5 to about 2% by weight, or such as from about 1 to about 2% by weight, and guar hydroxyproyltrimonium chloride is present in an amount of from about 0.1 to about 0.5% by weight, or such as from about 0.3 to about 0.5% by weight, including all ranges and subranges there-between, all weights being based on the total weight of the composition.

In yet other embodiments, the compositions of the present disclosure comprise polyquaternium-10 in an amount of about 2% by weight and guar hydroxyproyltrimonium chloride in an amount of about 0.5% by weight, all weights being based on the total weight of the composition.

### Cationic Surfactants

The at least one cationic surfactant of the present disclosure include, but are not limited to, cetrimonium chloride, quaternium-27, behenamidopropyl PG-dimonium chloride, hydroxyethyl tallowdimonium chloride, stearalkonium chloride, hydrogenated tallow alkyl trimethyl ammonium chloride, ditallow alkyl dimethyl ammonium chloride, and dihydrogenated tallow alkyl dimethyl ammonium chloride. Other cationic conditioners may include those that are naturally derived.

A preferred cationic surfactant of the present disclosure is cetrimonium chloride, commercially available from BASF under the tradename Dehyquart® A-OR and from Croda under the tradename Incroquat™ CTC-30-LQ-(MH).

The at least one cationic surfactant is present in the composition in an amount of from about 0.1 to about 20% by weight, such as from about 0.5 to about 10% by weight, and from about 1 to about 5% by weight, including all ranges and subranges there-between, based on the total weight of the composition.

### Aqueous Medium

The aqueous medium of the present disclosure may comprise water and mixtures of water and at least one cosmetically acceptable solvent chosen from organic solvents. Suitable organic solvents are typically C 1-C 4 lower alcohols and polyols alcohols. Examples of organic solvents include, but are not limited to, ethanol, isopropyl alcohol, benzyl alcohol and phenyl ethyl alcohol; glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, and also diethylene glycol alkyl ethers, such as diethylene glycol monoethyl ether and monobutyl ether; hydrocarbons such as straight chain hydrocarbons, mineral oil, isododecane, polybutene, hydrogenated polyisobutene, hydrogenated polydecene, polydecene, squalene, petrolatum and isoparaffins; and mixtures, thereof.

The aqueous medium can be present in the compositions of the present disclosure in the amount of about 95%, 90%, 80%, 70%, 60%, 50%, 40%, 30%, 20%, 10%, 5% by weight or less, based on the total weight of the composition. Additionally, aqueous medium can be present in the compositions of the present disclosure in the amount of from about 20% to about 95% by weight, or from about 50% to about 90% by weight, or from about 60% to about 80% by weight, based on the total weight of the compositions.

### Additional Surfactants

The compositions of the present disclosure can further comprise at least one surfactant selected from nonionic surfactants, anionic surfactants, amphoteric surfactants, zwitterionic surfactants and mixtures thereof.

Non-limiting examples of nonionic surfactants includes alkoxylated derivatives of the following: fatty alcohols, alkyl phenols, fatty acids, fatty acid esters and fatty acid amides, wherein the alkyl chain is in the C12-50 range, typically in the C16-40 range, more typically in the C24 to C40 range, and having from about 1 to about 110 alkoxy groups. The alkoxy groups are selected from the group consisting of C2-C6 oxides and their mixtures, with ethylene oxide, propylene oxide, and their mixtures being the typical alkoxides. The alkyl chain may be linear, branched, saturated, or unsaturated. Of these alkoxylated non-ionic surfactants, the alkoxylated alcohols are typical, and the ethoxylated alcohols and propoxylated alcohols are more typical. The alkoxylated alcohols may be used alone or in mixtures with those alkoxylated materials disclosed herein-above.

Commercially available nonionic surfactants are Brij® nonionic surfactants from Croda, Inc. Typically, Brij® is the condensation products of aliphatic alcohols with from about 1 to about 54 moles of ethylene oxide, the alkyl chain of the alcohol being typically a linear chain and having from about 8 to about 22 carbon atoms, for example, Brij® 72 (i.e., Steareth-2) and Brij® 76 (i.e., Steareth-10).

Also useful herein as nonionic surfactants are alkyl glycosides, which are the condensation products of long chain alcohols, which are the condensation products of long chain alcohols, e.g. C8-C30 alcohols, with sugar or starch polymers. These compounds can be represented by the formula (S)n —O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants are alkyl polyglucosides wherein S is a glucose moiety, R is a C8-C20 alkyl group, and n is an integer of from about 1 to about 9. Commercially available examples of these surfactants include decyl glucoside (available as Plantacare® 2000 UP) and lauryl glucoside (available as Plantacare® 1200 N and Plantacare® 1200 UP), all the above-identified polyglucosides are available from BASF. Also useful herein sucrose ester surfactants such as sucrose cocoate and sucrose laurate.

Other nonionic surfactants suitable for use in the present invention are glyceryl esters and polyglyceryl esters, including but not limited to, glyceryl monoesters, typically glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, and mixtures thereof, and polyglyceryl esters of C16-C22 saturated, unsaturated and branched chain fatty acids, such as polyglyceryl-4 isostearate, polyglyceryl-oleate, polyglyceryl-2 sesquioleate, triglyceryl diisostearate, diglyceryl monooleate, tetraglyceryl monooleate, and mixtures thereof.

Also useful herein as nonionic surfactants are sorbitan esters. Preferable are sorbitan esters of C16-C22 saturated, unsaturated and branched chain fatty acids. Because of the manner in which they are typically manufactured, these sorbitan esters usually comprise mixtures of mono-, di-, tri-, etc. esters. Representative examples of suitable sorbitan esters include sorbitan oleate (e.g., Span® 80), sorbitan sesquioleate (e.g., Span® 83 from Croda, Inc.,), sorbitan isostearate (e.g., Span® 120 from Croda, Inc. sorbitan stearate (e.g., Span® 60), sorbitan trioleate (e.g., Span® 85), sorbitan tristearate (e.g., Span® 65), and sorbitan palmitates (e.g., Span® 40). All the above-identified sorbitan esters are available from Croda, Inc. Also suitable for use as nonionic surfactants are alkoxylated derivatives of glyceryl esters, sorbitan esters, and alkyl polyglycosides, wherein the alkoxy groups are selected from the group consisting of C2-C6 oxides and their mixtures, with ethoxylated or propoxylated derivatives of these materials being typical. Nonlimiting examples of commercially available ethoxylated materials include TWEEN® (ethoxylated sorbitan mono-, di- and/or tri-esters of C12 to C18 fatty acids with an average degree of ethoxylation of from about 2 to 20).

Non-limiting examples of anionic surfactants include compounds in the classes known as alkyl sulfonates, alkyl ether sulfonates, sulfate esters of an alkylphenoxy polyoxyethylene ethanol, alpha-olefin sulfonates, beta alkyloxy alkene sulfonates, alkyl arylsulfonates, alkyl carbonates, alkyl ether carboxylates, fatty acids, succinamates, sulfosuccinates, sarcosinates, octoxynol or nonoxynol phosphates, taurates, fatty taurides, sulfated monoglycerides, isethionates and mixtures thereof. Specific examples of anionic surfactants include the ammonium, monoethanolamine, diethanolamine, triethanolamine, isopropylamine, sodium, potassium, lithium, or magnesium salts of dodecylbenzene-sulfonate, lauryl sulfosuccinate, lauryl ether carboxylate, lauryl sarcosinate, cocomethyl tauride, and sulfosuccinate half ester amide and mixtures thereof.

Non-limiting examples of amphoteric and zwitterionic surfactants include alkyl, alkyl dimethyl, alkylamido, alkyl amide, alkylamidopropyl, or alkyl dimethylammonium betaine; alky amidopropyl or alkyl sulfobetaine; alkyl, alkylampho, or alkylamphocarboxy glycinate; alkyl, or alkyl substituted imidazoline mono or dicarboxylate; sodium salts of alkyl mono- or dicarboxylates; alkyl beta amino acids; alkyl amidopropyl, or alkyl ether hydroxysultaine; alkyl amidopropyl dimethyl ammonia acetate; alkyl ampho mono-or diacetate; alkyl, or alkyl ampho, or alkyl imino dipropionate; alkyl amphopropionate; alkyl beta amino propionic acid; alkyl dipropionate; alkyl beta iminodipropionate; branched or n-alkyl dimethylamidopropionate; alkyl carboxylated propionate; alkyl, or methyl alkyl imidazoline; fluorinated alkyl amphoteric mixtures; and/or nonionic surfactants such as, but not limited to, alkyl, alkyl dimethyl, alkyl amidopropylamine, or bis 2-hydroxy ethyl alkyl amine oxides; alkanolamides; alkyl amides; polyoxyethylene glycol (PEG) of monoglycerides, of sorbitan esters, of branched or linear fatty alcohol ethers, of branched or linear fatty acid ethers, of thioethers; alkyl oxoalcohol PEG; PEG fatty esters; polyoxyethlyene glycol/polyoxpropylene glycol block copolymers; alkyl phenol PEG ethers; alkyl polyglucosides, or polysaccarides, polysiloxane polyethoxylene ether and mixtures thereof. Specific examples include cocamidopropyl betaine, lauramidopropyl betaine, coco/oleamidopropyl betaine, coco betaine, oleyl betaine, cocamidopropyl hydroxysultaine, tallowamidopropyl hydroxysultaine and dihydroxyethyl tallow glycinate and mixtures thereof.

In certain embodiments, the compositions of the present disclosure further comprise at least one nonionic surfactant.

The at least one surfactant is typically present in an amount from about 0.1 by weight to about 45% by weight, typically in an amount from about 5 by weight to about 30% by weight and more typically from about 10 by weight to 20% by weight, including all ranges and subranges there-between, based on the total weight of the composition.

### Lipophilic Compound

The compositions of the present disclosure can further comprise at least one lipophilic compound which can be chosen from oils, fatty esters, hydrocarbon oils, waxes, fatty acids and salts thereof, lipophilic vitamins and esters thereof, organic sunscreens, phospholipids, and mixtures thereof.

According to one embodiment, the at least one lipophilic compound is chosen from plant oils, hydrocarbon oils, synthetic oils, fatty acids having at least 12 carbon atoms, fatty esters and mixtures thereof.

According to another embodiment, the at least one lipophilic compound comprises fragrance oils.

According to other embodiments, the use of at least one lipophilic compound in the compositions of the present disclosure requires a solubilizer such as PEG-40 hydrogenated castor oil.

In preferred embodiments, the compositions of the present disclosure remain clear in appearance even when the at least one lipophilic compound is employed in said compositions.

### Auxiliary Ingredients

The composition may optionally contain at least one auxiliary ingredient. The auxiliary ingredients may include in particular, proteins, amino acids, skin and hair active agents, preservatives, pH adjusting agents, sequestering agents, and mixtures thereof.

As skin and hair active agents that may be used in the composition of the present disclosure, examples that may be mentioned include moisturizers, for example, protein hydrolysates and polyols such as glycerol, glycols, for instance polyethylene glycols, and sugar derivatives; natural and plant extracts; anti-inflammatory agents; antioxidants; anti-wrinkle agents; procyannidol oligomers; vitamins, for instancevitamin A (retinol), vitamin C (ascorbic acid), vitamin E (tocopherol), vitamin B5 (panthenol), vitamin B3 (niacinamide), derivatives of these vitamins (especially esters) and mixtures thereof; urea; caffeine; depigmenting agents such as kojic acid, hydroquinone and caffeic acid; salicylic acid and its derivatives; a-hydroxy acids such as lactic acid and glycolic acid and derivatives thereof; β-hydroxy acids, α-keto acids, β-keto acids, retinoids such as carotenoids and vitamin A derivatives; sunscreens; self-tanning agents; hydrocortisone; melatonin; algal, fungal, plant, yeast or bacterial extracts; enzymes; DHEA and its derivatives and metabolites; antibacterial active agents, for instance 2,4,4′-trichloro-2′-hydroxydi-phenyl ether (or Triclosan), 3, 4,4′-trichloro-carbanilide (or Triclocarban); mattifying agents and mixtures thereof.

Non-limiting examples of sunscreens include benzophenones, bornelone, butyl PABA, cinnamidopropyl trimethyl ammonium chloride, disodium distryrylbiphenyl disulfonate, PABA, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, ethylhexyl dimethyl PABA, red petrolatum, and mixtures thereof.

Non-limiting examples of preservatives include polyvinyl alcohol, phenoxyethanol, benzyl alcohol, methyl paraben, propyl paraben and mixtures thereof.

Non-limiting examples of pH adjusting agents include potassium acetate, sodium carbonate, sodium hydroxide, phosphoric acid, succinic acid, sodium citrate, citric acid, boric acid, lactic acid, sodium hydrogen carbonate and mixtures thereof.

The at least one auxiliary ingredient is present in the composition in a preferred amount of from about 0.001 to about 50% and more preferably from about 0.01 to about 20% by weight, based on the total weight of the composition.

According to certain embodiments, a pH reading of the compositions of the present disclosure ranges from about 3.5 to about 6.5, such as from about 4.5 to about 5.5

According to a preferred embodiment of the present disclosure, the composition is free of silicones.

According to another preferred embodiment of the present disclosure, the composition is clear in appearance.

According to yet another preferred embodiment of the present disclosure, the composition is free of fatty alcohols.

According to a particularly preferred embodiment of the present disclosure, the composition is free of silicones and is clear in appearance.

According to another particularly preferred embodiment of the present disclosure, the composition is free of silicones and fatty alcohols and is clear in appearance.

According to a preferred embodiment of the present disclosure, the composition is free of short chain alcohols.

The compositions of the present disclosure comprise aqueous cosmetic compositions.

The compositions described above are useful as compositions for conditioning and/or cleansing keratinous substrates such as hair and skin. These compositions include hair care products such as leave-in and rinse-out conditioners, shampoo/conditioner-in-one rinse-out products, hair and skin masque products, products for conditioning or cleansing/conditioning skin such as skin cleansers and personal hygiene products, and products for conditioning or cleansing/conditioning lips and nails.

For example, when the keratinous substrate being treated is hair, the compositions of the present disclosure impart good conditioning while providing a light-weight feel to the hair and styling benefits to hair. Such styling benefits may consist of allowing the hair to be shaped or styled without the need for additional styling or shaping products. Other properties, such as manageability, ease of combing, smoothness, softness, good/sleek feel, and styling, can also be provided by the compositions of the present disclosure to hair.

When the keratinous substrate is skin, the compositions may impart conditioning, including, long lasting conditioning, as well as smoothness, softness and moisturization to skin.

One embodiment of the present disclosure is a conditioning composition comprising, in an aqueous medium, at least one film forming polymer, at least one viscosity modifying agent, at least one cationic polymer, and at least one cationic surfactant. Preferably, said composition is clear in appearance and is free of silicones.

In preferred embodiments, the composition of the present disclosure is a clear composition used to provide conditioning benefits to hair and skin.

In particularly preferred embodiments, the composition of the present disclosure is a rinse-out conditioner for conditioning hair and skin. Said composition can be applied before or after cleansing or rinsing the hair or skin using water, or a shampoo, or a cleanser.

In other preferred embodiments, the composition of the present disclosure is a leave-in conditioner for conditioning hair and skin, wherein the composition can be applied after cleansing or rinsing the hair or skin using water, or a shampoo, or a cleanser.

The present invention is also directed to a process for conditioning a keratinous substrate involving contacting for conditioning a keratinous substrate comprising contacting keratinous substrates with the above-disclosed conditioning compositions.

### Method of Cosmetic Treatment or Use

The method or process of treatment to be provided will depend on the keratinous substrate being targeted and, consequently, the specific ingredients contained in the composition used to effectuate the treatment. One of ordinary skill in the art will easily be able to determine these variables.

A preferred embodiment of the present invention is a process for conditioning keratinous substrates such as hair and skin comprising contacting the keratinous substrates with a conditioning composition comprising, in an aqueous medium, at least one film forming polymer, at least one viscosity modifying agent, at least one cationic polymer, and at least one cationic surfactant. Preferably, said composition is clear in appearance and is free of silicones. The said composition may also be free of fatty alcohols.

According to at least one embodiment, such a process comprises applying to the hair or to the skin, an effective amount of the conditioning composition, and optionally rinsing it out after it has optionally been left on for a period of time.

When the composition according to the present disclosure is applied onto keratinous substrates before or after shampooing, it is optionally left on the hair for a time period ranging from 30 seconds to 5 minutes, and then optionally rinsed out with water.

An effective amount of the composition, typically from about 1 gram to about 50 grams, preferably from about 1 gram to about 20 grams of the composition, for cleansing hair, scalp, skin or other area of the body, is topically applied to the hair, scalp, skin or other area that has preferably been wetted, generally with water. Application to the hair typically includes working the composition, for example, a conditioner composition through the hair.

A preferred method for conditioning hair or skin comprises the steps of: (a) wetting the hair or skin with water, (b) applying an effective amount of the conditioning composition to the hair or skin, and (c) rinsing the composition from the hair or skin using water. These steps may be repeated as many times as desired to achieve the conditioning benefits sought.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1

### Formulation Examples

TABLE 1
Inventive conditioner formulas
(compositions were clear in appearance)

| INCI US | A | B | C |
|---|---|---|---|
| CITRIC ACID | 0.00667 | 0.00667 | 0.00667 |
| FRAGRANCE | 0.5 | 0.4 | 0.3 |
| PVP | 1 | 0.5 | 0.5 |
| POLYQUATERNIUM-10 | 0.91 | 0.91 | 0.91 |
| HYDROXYETHYLCELLULOSE | 0.5 | 0.65 | 0.65 |
| GUAR | 0.49 | 0.295 | 0.295 |
| HYDROXYPROPYLTRIMONIUM | | | |
| CHLORIDE | | | |
| PHENOXYETHANOL | 0.5 | 0.5 | 0.5 |
| PEG-12 DIMETHICONE | | | |
| WATER | 91.049 | 91.549 | 91.749 |
| GLYCERIN | 2.5 | 2.5 | 2.5 |
| PROPYLENE GLYCOL | 1 | 1 | 1 |
| PEG-40 HYDROGENATED | 0.5 | 0.4 | 0.3 |
| CASTOR OIL | | | |
| CETRIMONIUM CHLORIDE | 1 | 1 | 1 |
| PEG-45M | — | 0.25 | 0.25 |
| SODIUM ACETATE | 0.015 | 0.015 | 0.015 |
| ISOPROPYL ALCOHOL | 0.004 | 0.004 | 0.004 |
| SODIUM CHLORIDE | 0.015 | 0.015 | 0.015 |

### Procedure of Making:

Annex 1:

1. When guar hydroxypropyltrimonium chloride is employed in the inventive composition, this material was added to 30% of the total amount of water required for the composition. After the guar hydroxypropyltrimonium chloride was well hydrated, PVP was added to the mixture. Main Kettle:

1. 70% of the total amount of water required for the composition was placed in the kettle. Hydroxyethylcellulose was, added \and allowed to hydrate well. (hydration time can be shortened by heating the batch slightly to 45-50° C.) Polyquaternium-10 was added and mixed until fully hydrated.

2. Cetrimonium chloride, glycerin, and propylene glycol were each added and mixed to uniformity between additions.

3. Annex 1 was added to main kettle. The mixture was mixed to uniformity.

4. Phenoxyethanol and citric acid were added as preservatives.

5. The rest of the ingredients were added and mixed with the composition.

6. When fragrance is employed in the inventive composition, the fragrance was pre-solubilized in PEG-40 hydrogenated castor oil before it was added to the rest of the composition.

### Example 2

### Comparative Compositions

TABLE 2
Comparative conditioning formulas
(compositions were not clear or were very hazy or opaque in
appearance)

| INCI US | D | E | F | G | H | I | J |
|---|---|---|---|---|---|---|---|
| POLYQUATERNIUM- | 0.91 | 1.82 | 1.82 | 1.82 | 0.91 | 0.91 | 0.91 |
| 10 | | | | | | | |
| HYDROXYETHYLCELLULOSE | | | 0.5 | 0.5 | 1 | 2 | 1.2 |
| POTATO STARCH | | | | | | | 0.43 |
| MODIFIED | | | | | | | |
| POLYQUATERNIUM- | 1.4 | 1.4 | | | | | |
| 70 | | | | | | | |
| CHLORHEXIDINE | 0.03 | | | | | | |
| DIHYDROCHLORIDE | | | | | | | |
| PHENOXYETHANOL | 0.01 | 0.51 | 0.5 | 0.51 | 0.5 | 0.5 | 0.5 |
| PEG-12 | | | 1 | 1 | 2 | 2 | 2 |
| DIMETHICONE | | | | | | | |
| WATER | 96.256 | 94.842 | 92.342 | 91.842 | 91.586 | 91.556 | 91.926 |
| GLYCERIN | | | 3 | 3 | 2 | 2 | 2 |
| CETRIMONIUM | 0.6 | 0.6 | 0.5 | 1 | 1 | 1 | 1 |
| CHLORIDE | | | | | | | |
| BEHENTRIMONIUM | | | | | 0.79 | | |
| CHLORIDE | | | | | | | |
| HYDROXYPROPYLTRIMONIUM | 0.26 | 0.26 | 0.26 | 0.26 | | | |
| HYDROLYZED | | | | | | | |
| WHEAT PROTEIN | | | | | | | |
| SODIUM ACETATE | 0.015 | 0.03 | 0.03 | 0.03 | 0.015 | 0.015 | 0.015 |
| ISOPROPYL | 0.004 | 0.008 | 0.008 | 0.008 | 0.184 | 0.004 | 0.004 |
| ALCOHOL | | | | | | | |
| SODIUM CHLORIDE | 0.015 | 0.03 | 0.03 | 0.03 | 0.015 | 0.015 | 0.015 |
| DIPROPYLENE | 0.5 | 0.5 | | | | | |
| GLYCOL | | | | | | | |

The comparative compositions above were not clear in appearance; some of the compositions exhibited phase separation and/or were not desirable in terms of viscosity and rheology which affected their application and spreadability on hair.

TABLE 3
Traditional hair conditioner compositions
which employ fatty alcohols and which are not clear in
appearance.

| INCI US | Formula K | Formula L |
|---|---|---|
| BEHENTRIMONIUM CHLORIDE | 1.422 | 2.37 |
| CETRIMONIUM CHLORIDE | 0.016 | 0.012 |
| DODECENE | 0.0225 | 0.0225 |
| FATTY ESTERS | 0.25 | 0.5 |
| FATTY ALCOHOLS | 3.0275 | 3.5275 |
| FATTY CONDITIONING AGENTS | 0.35 | 0.2 |
| EMUSIFIERS | 0.28 | 0.26 |
| PEG-180 | 2 | |
| AMODIMETHICONE | 0.92 | 0.7 |
| HYDROXYETHYLCELLULOSE | 0.2 | |
| CITRIC ACID | 0.0625 | 0.0625 |
| ISOPROPYL ALCOHOL | 0.324 | 0.54 |
| VEGETABLE EXTRACT | 0.675 | 0.675 |
| VITAMIN | 0.25 | 0.25 |
| PRESERVATIVE | 0.22 | 0.22 |
| FRAGRANCE | 0.5 | 0.5 |
| WATER | 89.48103 | 90.16 |

### Example 3

### Salon Tests

Inventive and comparative formulas were applied onto the hair of human models in a salon by licensed professionals. Half-head testing was performed with an inventive formula applied to hair on one side of the head and a comparative applied to the hair on the other side of the head.

Salon test: Inventive formula (Formula C in Table 1) versus a comparative traditional commercial formula (Pantene Flat to Volume Conditioner product: Water, Cetyl Alcohol, Stearamidopropyl Dimethylamine, Stearyl Alcohol, Quaternium 18, Fragrance, Bis Aminopropyl Dimethicone, Benzyl Alcohol, Cetearyl Alcohol, Hydroxypropyl Guar, Oleyl Alcohol, Glyceryl Stearate, Citric Acid, EDTA (Ethylenediamine-Tetra-Acetic Acid), Polysorbate 60, Panthenol, Panthenyl Ethyl Ether, Methylchloroisothiazolinone, Methylisothiazolinone.

The inventive formula demonstrated better cosmetic attributes of discipline (manageability/reduced fly aways at the ends of the hair fibers), shine, smoothness, volume, and shapeability compared to the comparative formula.

### Example 4

### Transmittance Measurements

Transmittance measurements were employed in order to evaluate the clarity of the inventive compositions. It was found that the inventive compositions which were clear in appearance had transmittance values ranging from 80 to 100% as measured on a spectrophotometer (Agilent UV-Visible ChemStation System, model 8453).

The foregoing description illustrates and describes the present disclosure. Additionally, the disclosure shows and describes only the preferred embodiments of the disclosure, but, as mentioned above, it is to be understood that it is capable of changes or modifications within the scope of the concept as expressed herein, commensurate with the above teachings and/or skill or knowledge of the relevant art. The embodiments described hereinabove are further intended to explain best modes known of practicing the invention and to enable others skilled in the art to utilize the disclosure in such, or other, embodiments and with the various modification required by the particular applications or uses disclosed herein. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

What is claimed:

1. A conditioning composition comprising:
(a) from about 0.5% to about 1% by weight of at least one nonionic film forming polymer selected from the group consisting of polyvinylpyrrolidone, vinyl pyrrolidone/vinyl acetate copolymer, and mixtures thereof;
(b) from about 0.5% to about 1.2% by weight of at least one viscosity modifying agent selected from the group consisting of hydroxyethylcellulose, cetyl hydroxyethylcellulose, guar gum, xanthan gum and mixtures thereof;
(c) from about 0.5% to about 2% by weight of at least one cationic polymer selected from the group consisting of polyquaternium-10, polyquaternium-70, guar hydroxypropyltrimonium chloride, and mixtures thereof;
(d) from about 1% to about 5% by weight of at least one cationic surfactant selected from the group consisting of cetrimonium chloride, quaternium-27, behenamidopropyl PG-dimonium chloride, hydroxyethyl tallowdimonium chloride, stearalkonium chloride, hydrogenated tallow alkyl trimethyl ammonium chloride, and mixtures thereof;
(e) from about 90% to about 95% by weight of an aqueous medium comprising water and optionally an organic solvent, said aqueous medium comprising at least about 90% water;
wherein the composition contains 0% silicones, and 0% fatty alcohols, less than 0.1% short chain alcohols, and 0% of ethoxylated cholesterol or laneth-16 lanolin derivatives, by weight, all weights being based on the total weight of the composition, has a pH of from about 4.5 to about 5.5, and has a light transmittance value of about 70% or more.

2-14. (canceled)

15. A process for conditioning, hair comprising contacting the hair with the composition of claim 1.

16. The process of claim 15, wherein the process imparts styling benefits to the hair.

17. A conditioning composition comprising:
(a) from about 0.5 to about 1% by weight of at least one nonionic film forming polymer selected from the group consisting of polyvinylpyrrolidone, vinyl pyrrolidone/vinyl acetate copolymer, and mixtures thereof;
(b) from about 0.1 to about 1.5% by weight of at least one viscosity modifying agent selected from the group consisting of hydroxyethylcellulose, cetyl hydroxyethylcellulose, guar gum, xanthan gum and mixtures thereof;
(c) from about 0.1 to about 4 0.5% to about 1% by weight of at least one cationic polymer selected from the group consisting of polyquaternium-10, polyquaternium-70, guar hydroxypropyltrimonium chloride, and mixtures thereof;
(d) from about 1 to about 5% by weight of at least one cationic surfactant selected from the group consisting of cetrimonium chloride, quaternium-27, behenamidopropyl PG-dimonium chloride, hydroxyethyl tallowdimonium chloride, stearalkonium chloride, hydrogenated tallow alkyl trimethyl ammonium chloride, and mixtures thereof;
(e) from about 90% to about 95% of an aqueous medium comprising water and optionally an organic solvent, said aqueous medium comprising at least about 90% water;
(f) at least one surfactant chosen from the group consisting of anionic surfactants, amphoteric surfactants and zwitterionic surfactants;
wherein the composition contain 0% silicones, 0% fatty alcohols, less than 0.1% short chain alcohols, and 0% of ethoxylated cholesterol or laneth-16 lanolin derivatives, by weight, all weights being based on the total weight of the composition, has a pH of from about 4.5% to about 5.5% and has a light transmittance value of about 70% or more.

18. (canceled)

19. A process for conditioning hair comprising contacting the keratinous substrate with the composition of claim 17.

20. The composition of claim 17 further comprising a fragrance oil and a solubilizer.

21. The composition of claim 20 wherein the solubilizer is PEG-40 hydrogenated castor oil.

22. The composition of claim 1 wherein the organic solvent is selected from the group consisting of ethanol, isopropyl alcohol, benzyl alcohol, phenyl ethyl alcohol, propylene glycol, hexylene glycol, ethylene glycol monomethyl, monoethyl ether, monobutyl ether, propylene glycol, propylene glycol monomethyl ether, butylene glycol, dipropylene glycol, diethylene glycol monoethyl ether, monobutyl ether, mineral oil, isododecane, polybutene, hydrogenated polyisobutene, hydrogenated polydecene, polydecene, squalene, petrolatum and isoparaffins, and mixtures, thereof.

23. The composition of claim 22 wherein the organic solvent is isopropyl alcohol.

24. The composition of claim 23 further comprising propylene glycol.

25. The composition of claim 24 further comprising glycerin.

26. The composition of claim 25 further comprising PEG-40 Hydrogenated castor oil.

27. (canceled)

28. The composition of claim 1 comprising two cationic polymers selected from polyquaternium-10, polyquaternium-70, and guar hydroxypropyltrimonium chloride.

29. The composition of claim 28 comprising from about 0.5 to about 2% by weight of polyquaternium-10 and from about 0.1 to about 0.5% by weight of guar hydroxypropyltrimonium chloride, all weights being based on the total weight of the composition.

30. The composition of claim 17 comprising two cationic polymers selected from polyquaternium-10, polyquaternium-70, and guar hydroxypropyltrimonium chloride.

31. The composition of claim 30 comprising from about 0.5 to about 2% by weight of polyquaternium-10 and from about 0.1 to about 0.5% by weight of guar hydroxypropyltrimonium chloride, all weights being based on the total weight of the composition.

32. The composition of claim 17 wherein the organic solvent is isopropyl alcohol.

33. The composition of claim 32 further comprising propylene glycol.

34. A conditioning composition consisting essentially of
(a) from about 0.5% to about 1% by weight of at least one nonionic film forming polymer selected from polyvinylpyrrolidone;
(b) from about 0.5% to about 1% by weight of at least one viscosity modifying agent selected from hydroxyethylcellulose;
(c) from about 0.3% to about 0.5% by weight of a cationic polymer selected from guar hydroxypropyltrimonium chloride and about 1% by weight of a cationic polymer selected from polyquaternium-10;
(d) about 1% by weight of a cationic surfactant selected from cetrimonium chloride;
(e) from about 91% to about 92% by weight water and about 0.004% by weight propylene glycol;
(f) about 2.5% by weight glycerin;
(g) from about 0.3% to about 0.5% by weight PEG-40 hydrogenated castor oil; and
(h) about 0.0067% by weight of a pH adjuster;
by weight, all weights being based on the total weight of the composition, has a pH of from about 4.5 to about 5.5, and has a light transmittance value of about 70% or more.
